(19) United States
(12) Patent Application Publication
(10) Pub. No.: US 2025/0367189 A1
(43) Pub. Date: Dec. 4, 2025

(54) 2-BROMO-LYSERGIC ACID DIETHYLAMIDE FOR SUBSTANCE ABUSE

(71) Applicant: Ceruvia Lifesciences LLC, Greenwich, CT (US)

(72) Inventor: Carey TURNBULL, Greenwich, CT (US)

(73) Assignee: Ceruvia Lifesciences LLC, Greenwich, CT (US)

(21) Appl. No.: 19/302,500

(22) Filed: Aug. 18, 2025

Related U.S. Application Data
(63) Continuation-in-part of application No. 17/913,471, filed on Sep. 22, 2022.
(60) Provisional application No. 62/994,590, filed on Mar. 25, 2020.

(51) Int. Cl. A61K 31/48; A61P 25/32

(52) U.S. Cl. CPC A61K 31/48; A61P 25/32

(57) ABSTRACT

A method of treating substance abuses in an individual, by administering a first dose of 2-bromo-lysergic acid diethylamide (BOL-148) to an individual, producing an additive effect by administering a second dose of BOL-148 to the individual, and reducing use of the substance.

### BACKGROUND OF THE INVENTION

### 1. Technical Field

The present invention relates to compositions and methods for treating substance abuses, and especially alcohol use disorder (AUD). More specifically, the present invention relates to methods of using 2-bromo-lysergic acid diethylamide (BOL-148) in treating substance abuses.

### 2. Background Art

Alcohol use disorder (AUD) is a disease that is generally characterized by compulsive alcohol use, loss of control over alcohol intake, and a negative emotional state when not using. Of all drugs of abuse, alcohol has been ranked as the most damaging when harms to the user and others are considered, including mortality, morbidity, dependence, loss of function, social impairment, injury, psychological damage, crime, environmental impacts, and economic costs. This disease affects about 15 million people in the United States (up to 12% of the population at some point in their lifetime), including adolescents and adults, as well as their families. The economic cost of alcohol use is staggering, estimated at $185 billion yearly in the US alone, compared to $158 billion for nicotine and $280 billion for illicit drugs (includes costs related to criminalization). The effects of currently available treatments for AUD remain disappointingly small, and attempts to improve outcomes through combination therapy (multiple pharmacotherapies or pharmacotherapy plus behavioral therapy) have been largely disappointing; approximately one person achieves abstinence or avoids relapse for every nine people treated with the most effective FDA-approved pharmacotherapies for AUD (Rosner, S., et al.)

Several types of treatment are available for AUD. Behavioral treatments such as counseling can help change an individual's drinking behavior, such as providing coping mechanisms and suggesting the individual avoid triggers that cause drinking. There are also many support groups such as Alcoholics Anonymous that provide peer support to help individuals to stop drinking. Medications can also be prescribed to help stop or reduce drinking.

Naltrexone is drug that acts as a competitive antagonist at the μ-opioid receptor that is prescribed to manage alcohol or opioid dependence. Naltrexone can decrease the amount and frequency of drinking; however, it seems to only have a modest effect on AUD. In treating AUD, it is taken orally about an hour before drinking to avoid side effects. Naltrexone blocks the positive-reinforcement effects of alcohol. It can decrease cravings for opioids as well after a few weeks and can decrease risk of overdose. For this indication, naltrexone is injected once a month. There are several side effects, including diarrhea, abdominal cramping, liver damage, trouble sleeping, anxiety, nausea, and headaches.

Acamprosate stabilizes chemical signaling in the brain that would otherwise be disrupted by alcohol withdrawal. It has not been found to be effective alone, and requires psychosocial support. Side effects include allergic reactions, abnormal heart rhythms, low or high blood pressure, diarrhea, headaches, insomnia, and impotence. Major side effects can include suicidal behavior, major depressive disorder, and kidney failure.

Disulfiram produces an acute sensitivity to ethanol by inhibiting the enzyme acetaldehyde dehydrogenase. This effectively produces a hangover effect immediately after drinking. Side effects include flushing, throbbing in the head and neck, headaches, respiratory difficulty, nausea, vomiting, sweating, thirst, chest pain, palpitations, dyspnea, hyperventilation, fast heart rate, low blood pressure, fainting, uneasiness, weakness, vertigo, blurred vision, and confusion. Severe side effects include respiratory depression, cardiovascular collapse, abnormal heart rhythms, heart attack, acute congestive heart failure, unconsciousness, convulsions, and death.

Other drugs that are used for other indications can also be helpful in treating AUD, including varenicline (anti-smoking), gabapentin (pain and epilepsy), topiramate (anti-epileptic).

Psychedelic drugs (a subtype of hallucinogens) have also been investigated in treating AUD or other addictions. Extensive research was conducted on the use of the prototypical classic psychedelic lysergic acid diethylamide (LSD) in the treatment of alcoholism from roughly 1953-1970 (Abuzzahab and Anderson, Halpern, Mangini, Dyck, and Grinspoon and Balakar). A meta-analysis published in 2012 by Krebs and Johansen examined 6 randomized trials published between 1966-1970 that met basic standards for modern clinical trials, and determined that results consistently favored LSD for the treatment of alcohol dependence. The trials included a total of 325 participants treated with a single high-dose LSD session, as well as 211 participants that received control treatment, but were otherwise dissimilar; with varying sample sizes, LSD doses, control conditions, and preparation, monitoring, and debriefing for the LSD treatment sessions. Nonetheless, at the first post-treatment follow-up, odds of improvement across all six trial were nearly twice as high in participants treated with LSD (OR=1.96, 95% CI=1.36-2.84; Z=3.59, p=0.0003).

Although clinical research on psychedelics stalled for just over 20 years following enactment of the Controlled Substances Act in 1970, its resurgence is well underway with nearly 30 years of relevant modern research completed. Contemporary clinical trials with psychedelics have focused mainly on psilocybin (rather than LSD), due to its relatively short-lived acute effects and excellent safety profile. Psilocybin has shown promise for treatment of obsessive compulsive disorder, major depressive disorder, anxiety, and depression related to cancer diagnoses, cluster headache, smoking cessation, and AUD. Among the many indications that have been explored as potential therapeutic targets of classic psychedelics, AUD emerges as the leader in terms of number of participants treated in modern clinical trials and empirical data generated.

Bogenschutz, et al. (Journal of Psychopharmacology 2015, Vol. 29 (3) 289-299) examined in a pilot study ten volunteers with DSM-IV alcohol dependence who received orally administered psilocybin in one or two supervised sessions in addition to Motivational Enhancement Therapy and therapy sessions devoted to preparation for and debriefing from the psilocybin sessions. Participants' responses to psilocybin were qualitatively similar to those described in other populations. Abstinence did not increase significantly in the first 4 weeks of treatment (when participants had not yet received psilocybin), but increased significantly following psilocybin administration (p<0.05). Gains were largely maintained at follow-up to 36 weeks.

Dipropyltryptamine (DPT) has also been investigated in the treatment of alcoholism. In a single-group pilot study involving 51 participants, Grof et al. (1973) reported highly significant improvement in clinical outcomes including abstinence among the 47 participants (92%) who received between 1 and 6 DPT (mean 1.9) sessions and completed follow-up at 6 months. However, a subsequent trial comparing DPT treatment with conventional treatment found no significant differences between DPT-treated participants and the other groups in clinical outcomes assessed at 6 month follow-up, and conventional treatment group members assessed at 12 months reported better drinking outcomes and social functioning than the other two groups. Therefore, it is not clear that all psychedelics can treat alcohol abuse.

Alper, et al. (Frontiers in Pharmacology, 31 Aug. 2018) showed that mice treated with 25 μg/kg LSD had reduced ethanol consumption (group mean reduction of 17.9%) relative to controls. This effect was shown for 46 days of observation. However, LSD is a schedule I drug that is not approved for any medical use, making it impossible to prescribe to those who need treatment of AUD. Moreover, LSD induces powerful temporary consciousness-altering (‘psychedelic’) effects that render its delivery to human patients logistically challenging. The mechanism of action underlying this LSD-induced reduction in alcohol preference remains unclear. Existing (approved) treatments for AUD described above are not psychedelic, and their mechanisms of action are each distinct from effects known to be produced by psychedelic compounds.

LSD has known effects at several types of receptors, such as dopamine receptors, adrenergic receptors, and serotonin receptors (5HT) 1A, 2A, 5C, and 6. LSD is a potent agonist of 5HT1A receptors (Aghajanian G K. (1995): Electrophysiology of serotonin receptor subtypes and signal transduction mechanisms. In Bloom F E, Kupfer D J (eds), Psychopharmacology: The Fourth Generation of Progress. New York, Raven Press, pp 451-460), a partial agonist of 5HT2A receptors (i.e. not as strong of an agonist, Marek, et al., J Pharmacol Exp Ther. 1996 September; 278 (3): 1373-82), and a partial agonist of dopamine receptors (Giacomelli, et al. Life Sci. 1998; 63 (3): 215-22). 5HT2A receptors are involved in psychedelic and hallucinogenic effects. Pierce, et al. state that “data from radioligand binding, cellular, smooth muscle, and behavioral studies . . . suggest that d-LSD is a potent 5-HT2 antagonist.” (Neuropsychopharmacology. October-December 1990; 3 (5-6): 503-8.) The belief that LSD was an antagonist likely was because it is actually a partial agonist at certain receptors versus an agonist. This shows that there was much unknown about LSD in early studies, and that statements and experiments done in the past are not necessarily predictive of knowledge at the current time.

2-Bromo-LSD (also called BOL-148) is a derivative of LSD that is inactive as a psychedelic (and not scheduled) but also has an effect on 5-HT receptors as a neutral antagonist (i.e. it occupies a receptor binding site but doesn't induce constitutive receptor activity). BOL-148 having halogenation at the 2-position of LSD provides 5HT2A antagonist activity (Sagar et al. A Brief Review of Chemistry and Pharmacology of Lysergic Acid Diethylamide. Research J. Pharm. and Tech 2017; 10 (12): 4415-4422). Jadhav, et al. (A Brief Review of Chemistry and Pharmacology of Lysergic Acid Diethylamide. Research J. Pharm. and Tech 2017; 10 (12): 4415-4422.) state that “Pre-treatment with BOL-148, a non-hallucinogenic congener of LSD with serotonin antagonist properties like LSD, will not block the effects of LSD” with regards to experiments in rats. This statement should be interpreted that LSD has a higher affinity for 5-HT2A receptors than BOL-148 and can displace BOL from occupied receptors in rats, as Jadhav, et al. clearly state that LSD itself is a 5-HT2A agonist. Nichols, et al. states regarding humans that “Although virtually no work has been done with BOL-148 since the early 1970s, it was demonstrated early on that it could block the effects of LSD in humans” (WIREs Membr Transp Signal 2012, 1:559-579.)

U.S. Pat. No. 8,883,808 to Bonavanture, et al. discloses administering effective amounts of an SRI and a 5-HT7 receptor antagonist to a subject to treat serotonin-mediated diseases and conditions (or their associated symptoms) that are mediated through increasing the release of serotonin, inhibiting its reuptake, or both, or by increasing activity of serotonergic neurons, such as those associated with aberrant 5-HT7 receptor levels or serotonin reuptake activity or function. The 5-HT7 antagonist can be 2-Br LSD (BOL-148). One such disease can be alcohol abuse. There is no disclosure of using the 5-HT7 receptor antagonist without being in combination with an SRI. There is much support for the involvement of 5-HT2A and downstream neuroplasticity-related events that it can trigger underlying anti-addictive effects of other 2A agonists (eg psilocybin), that the effects of any one of these compounds are unlikely to be due to actions at 5HT7 alone.

Assays can be performed to find agents that bind or inhibit binding to serotonin receptors. For example, U.S. Pat. No. 6,844,190 to Sibley, et al. discloses isolating mammalian serotonin receptor protein St-B17 and using it in an assay for screening drug candidates. Br-LSD (BOL-148) was tested in displacing LSD.

Contrary to the usual course of drug development, clinical trials with classic psychedelics have preceded preclinical research. Largely due to constraints introduced by the Controlled Substances Act and the placement of all classic psychedelics into the highly restrictive Schedule I class, reserved for substances with high addictive potential and no clinical value, basic science research on classic psychedelics has entirely avoided questions related to mechanism of clinical action. LSD is among the most well-studied pharmacological substances and has played a central role in modern neuroscience and development of other pharmacotherapies, but almost nothing is known about the potential mechanism by which a single exposure to a classic psychedelic, such as LSD or psilocybin, could produce immediate and persistent improvements in clinical diagnoses such as AUD.

Despite promising results from human clinical trials using classic psychedelics for treatment of addiction published animal studies on the effects of classic psychedelics on self-administration of alcohol or other drugs of abuse are extremely limited. The prototypical classical psychedelic, 2,5-dimethoxy-4-iodoamphetamine (DOI) has been shown to reduce alcohol preference in rats (Maurel, et al.), but the generalizability of this finding to LSD or psilocybin, and its relevance to humans, is questionable. A single contemporary study found that acute administration of 50 ug/kg LSD produced a robust and lasting reduction in alcohol consumption and alcohol preference in C57BL/6J mice (Alper, et al.). There are no published reports on the mechanism by which classic psychedelics produce lasting reductions in alcohol preference or the reinforcing effects of alcohol.

Results from human clinical trials have shown that measures of the intensity and/or mystical-type content of the acute consciousness-altering effects of psilocybin are predictive of clinical improvement in studies of AUD and cancer-related distress (Ross, et al., Griffiths, et al., Bogenschutz, et al.). Given these findings, there are three possibilities regarding the relationship between the acute consciousness-altering effects of classic psychedelics and their clinical effects: 1) Clinical mechanism that is positively related to, and dependent on, acute psychedelic effects; 2) Clinical mechanism that is positively related to, but not dependent on, acute psychedelic effects; or 3) Additive or interactive clinical mechanisms with mixed dependence on acute psychedelic effects.

Findings from Alper, et al. support the possibility that the acute consciousness-altering effects of classic psychedelics may not be necessary for their impact on alcohol preference. Because the study was carried out in rodents, it is impossible to judge the hallucinogenicity of any of the treatments; however, the high dose of LSD that reduced alcohol preference in this study (50 ug/kg) is well below the range of doses that reliably produce accurate responding in a discrimination task or behavioral indicators of hallucinogenisis in rodents, such as the head twitch response (170-200 ug/kg) (Halberstadt, Benneyworth, et al., and Winter, et al). However, from these studies, it cannot be concluded that psychedelic effects are needed in treating AUD, or that a non-psychedelic compound would work effectively.

There have been no studies previously performed investigating treating AUD with BOL-148 or providing dosing in humans. While many treatments are available for AUD, there remains a need for a treatment with fewer side effects as well as for a medication that is readily available.

### SUMMARY OF THE INVENTION

The present invention provides for a method of treating substance abuses in an individual, by administering a first dose of 2-bromo-lysergic acid diethylamide (BOL-148) to an individual, producing an additive effect by administering a second dose of BOL-148 to the individual, and reducing use of the substance.

### DESCRIPTION OF THE DRAWINGS

Other advantages of the present invention are readily appreciated as the same becomes better understood by reference to the following detailed description when considered in connection with the accompanying drawings wherein:

FIG. 1 is a schematic of a study timeline;

FIG. 2A is a line graph showing alcohol consumption by weight, FIG. 2B is a condensed line graph showing alcohol consumption by weight, and FIG. 2C is a bar graph showing alcohol consumption by weight;

FIG. 3A is a line graph showing alcohol preference by weight, FIG. 3B is a condensed line graph showing alcohol preference by weight, and FIG. 3C is a bar graph showing alcohol preference by weight; and

FIG. 4A is a bar graph showing within-group reduction in alcohol preference with BOL-148 and FIG. 4B is a bar graph showing within-group reduction in alcohol preference with LSD.

### DETAILED DESCRIPTION OF THE INVENTION

The present invention generally provides for a method of treating substance abuses in an individual, by administering 2-bromo-lysergic acid diethylamide (BOL-148) to an individual, and reducing use of the substance. More specifically, a first dose of BOL-148 is administered to the individual, a second dose is administered to the individual, and this second dose provides an additive effect of the BOL-148 which causes reduction of use of the substance. BOL-148 can work in reducing substance use in a similar manner to LSD by its effects at the 5HT2A receptor, further described below. However, unlike LSD, BOL-148 has non-psychedelic pharmacological properties which allow it to work in treating substance use.

Reducing use of the substance can include reducing frequency of use, reducing an amount of substance used, and/or stopping use of the substance.

The substance abuse can be, but is not limited to alcohol, cocaine, nicotine, opiates, or any other substance that can be abused. In other words, an individual can be treated for using substances such as cocaine, nicotine, opiates, alcohol, morphine, methamphetamine, or other substances. Behavioral disorders can also be treated in the present invention.

Doses of BOL-148 used in an initial rodent study are 0.03 mg/kg and 3.0 mg/kg, which is equivalent to 0.75 μg (7.5 mg×10⁻⁴ mg) and 75 μg (0.075 mg) for an average mouse weighing 25 g (0.025 kg). For an average human weighing 70 kg, equivalent doses are 2.1 mg and 210 mg, respectively. Therefore, a dose of 2.1 to 210 mg can be used in humans. The lower dose of BOL-148 used in this trial is equivalent (0.03 mg/kg) on a per-weight basis to a dose shown to be effective at reducing cluster headaches when delivered daily to human participants in an open-label compassionate use trial (Karst et al., Cephalalgia. 2010 September; 30 (9): 1140-4).

Administration can be hourly, daily, weekly, bi-weekly, or longer periods of time. More specifically, administering a second dose can be performed at a time period of an hour, multiple hours, a day, a week, or two weeks after said step of administering a first dose. Subsequent doses can be administered at a same or different period of time from the first two doses.

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, intratonsillar, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

The doses can be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

### Materials and Methods

### Animals

All experiments will be conducted in adult male C57BL/6J mice (10-18 weeks old; Jackson Laboratory, United States). Mice will be given unlimited access to standard mouse chow and water throughout the entire study.

### Ethanol Drinking Behavior

The effect of BOL-148 on ethanol consumption will be assessed using a two-bottle choice drinking paradigm. Mice will be housed singly and habituated to reverse light-dark cycle for at least a week. The mice will be then exposed to ethanol to develop preference with two bottles containing water and 20% ethanol offered for 24 hours in the beginning of the dark phase for five days a week (Monday through Friday) for 4 weeks. The mice will be then divided into three groups of equal ethanol intake based on the amount of ethanol consumed on the day before administration of either saline or BOL-148 (0.3 mg/kg, or 3.0 mg/kg IP; approximately 9 μg and 90 μg for an average 30 g mouse) once before the onset of the dark cycle. BOL-148 will be supplied by the NIDA Drug Supply Program, Bethesda, MD, United States. Water bottles will be replaced with bottles containing water and ethanol after 10 minutes of treatment. Amounts of ethanol and water consumption will be measured every 24 hours. The positions of water and ethanol bottles will be alternated every day to avoid place preference. Two bottles containing water and 20% ethanol will be placed in a cage without a mouse to control for spillage and evaporation.

### Locomotor Activity

Ambulatory activity will be measured as ambulatory counts (interruption of the total number of beams, on both the x and y-axis) with an infrared beam-based activity sensor (ATM3; Columbus Instrument, Columbus, OH, United States) over a 24-hour period beginning at the dark cycle at 1 and 8 days after the administration of 3.0 mg/kg BOL-148 or saline to ethanol-naïve mice.

### Statistical Analysis

Data will be analyzed using SPSS version 25 for Windows (IBM Corp., Armonk, N.Y., USA; released 2017), using repeated measures ANOVA (MIXED command), including treatment group, time (Day 1-42), and the interaction between time and treatment group as independent variables. Data will be analyzed separately for each of 3 dependent variables: ethanol consumption, ethanol preference (ethanol consumption/total fluid consumption), and total fluid consumption. Alpha level will be set at 0.05 for all comparisons.

### Example 2

This study directly addresses the question of whether the acute psychedelic effects of LSD are necessary for it to alter ethanol preference in alcohol-preferring mice. The study replicates and extend the findings from Alper et al. to explore the following aims:

### Aim 1: Determine Whether LSD-Induced Reduction in Alcohol Preference is Cumulative

Hypothesis 1a: Alcohol preference will be reduced following single administration of 50 ug/kg LSD, relative to vehicle, treatment.
Hypothesis 1b: Alcohol preference will be further reduced following a second administration of 50 ug/kg LSD, relative to vehicle, treatment.
   Aim 2: Determine Whether Psychedelic Effects of Classic Psychedelics are Necessary for their Impact on Alcohol Preference
Hypothesis 2a: Alcohol preference will be reduced following a single administration of 3.0 mg/kg BOL-148, a non-psychedelic analog of LSD, relative to vehicle treatment.
Hypothesis 2b: Only partial reduction in alcohol preference will be achieved by BOL-148, relative to LSD treatment.

### Approach

### Animals

Twenty-five adult (12-30-week old), male fosTRAP2×Ai14 mice were bred in-house. Generation of the fosTRAP2 and FosTRAP lines were previously described (Guenthner, et al.). fosTRAP2 mice were crossed with Ai14 mice to obtain the mice used in this study. Mice were single-housed on a reverse light-dark cycle and given unlimited access to standard mouse chow and water. Animals were given a week to habituate to single housing, after which they were given free access to 2 bottles containing water and 20% ethanol for 24-hour periods beginning at the start of their dark cycle, 5 days per week (Monday-Friday) for 4 weeks (ethanol exposure). Amount of water and ethanol consumed (by volume and weight) were measured for each mouse across the final 5 days of the ethanol exposure period, and ethanol preference was counterbalanced across treatment group by the mean ethanol preference by weight (corrected for body weight) during this period.

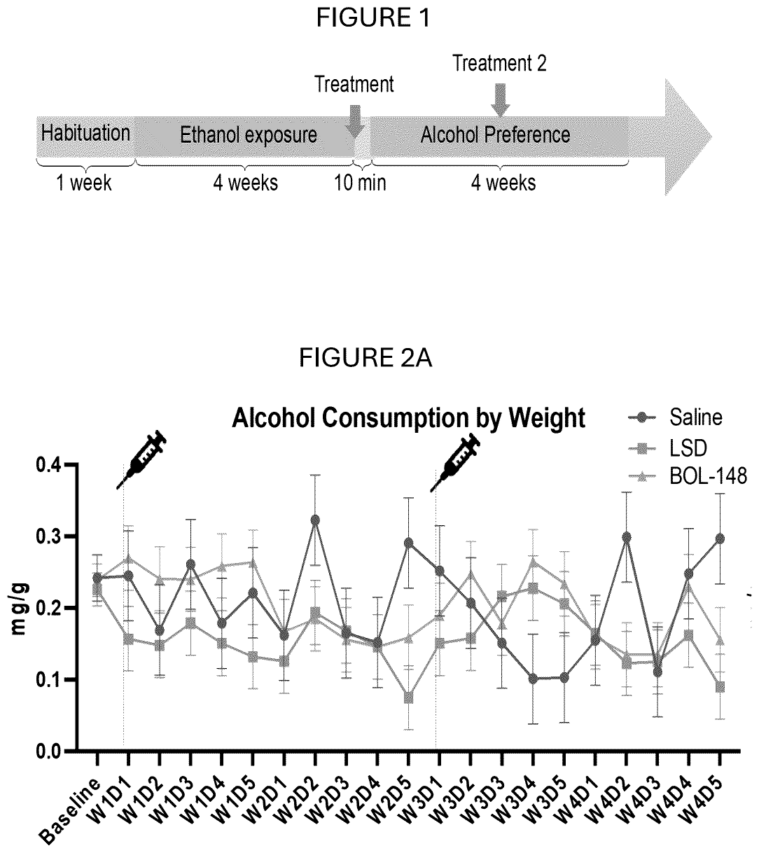

Following 4 weeks of ethanol exposure, animals were administered one of 3 treatments via a single intraperitoneal injection 10 minutes prior to onset of the dark cycle/day one in a five-day cycle of alcohol exposure: saline, LSD 50 ug/kg, or BOL-148 3.0 mg/kg. Following administration of treatment, animals were placed back into their home cages with free access to standard mouse chow and water. After 10 minutes, 12 animals from each treatment group were given free access to 2 bottles containing water and 20% ethanol for 24 hours daily for 4 weeks (alcohol preference). Amount of water and ethanol consumed (by volume and weight) were measured daily at the end of the light cycle. Positions of bottles containing water and 20% ethanol were alternated daily to prevent the formation of place preference. An additional 2-4 animals per treatment group were given free access to standard mouse chow and water, and ambulatory activity as well as head-twitching behavior (thought to correspond with hallucinogenicity of substances in mice), was measured for 35 minutes following treatment. FIG. 1 shows a schematic of the study timeline.

At the end of the experiment, animals were sacrificed via decapitation under isofluraneanesthesia. All procedures were approved by the NYULMC Institutional Animal Care and Use Committee (IACUC), and adhered to Guidelines for the Care and Use of Laboratory Animals and NIH standards.

### Statistical Analysis

For all hypotheses, data were analyzed using Mixed Models for Repeated Measures (MMRM), including fixed effects of Treatment (Saline, BOL, LSD), Week (1-4), Day (1-5), and two- and three-way interactions between Treatment and Day/Week. Repeated Effects were represented as a nested within-subjects variable (days within weeks). Data were analyzed separately for each of 3 dependent variables: ethanol consumption (mg and ml consumed), ethanol preference (ethanol consumption/total fluid consumption), and total fluid consumption (mg and ml consumed). For hypothesis 1a, a priori between-group comparisons are performed to contrast ethanol consumption, ethanol preference, and total fluid consumption between the vehicle- and LSD-treated groups. For hypothesis 1b, a priori between-group comparisons are performed to contrast ethanol consumption, ethanol preference, and total fluid consumption between the LSD-treated group at follow-up weeks 1 versus 3. For hypothesis 2a, a priori between-group comparisons are performed to contrast ethanol consumption, ethanol preference, and total fluid consumption between the vehicle- and BOL-148-treated groups. For hypothesis 2b, a priori between-group comparisons are performed to contrast ethanol consumption, ethanol preference, and total fluid consumption between the LSD- and BOL-148-treated groups. For each hypothesis, with 3 groups of 5-10 animals (total n=25) and 20 days of repeated measurements (autocorrelation=0.5), there is power of 0.8 to detect moderately-sized effects of approximately d=0.3 at alpha=0.05.

### Results

### Ethanol Consumption

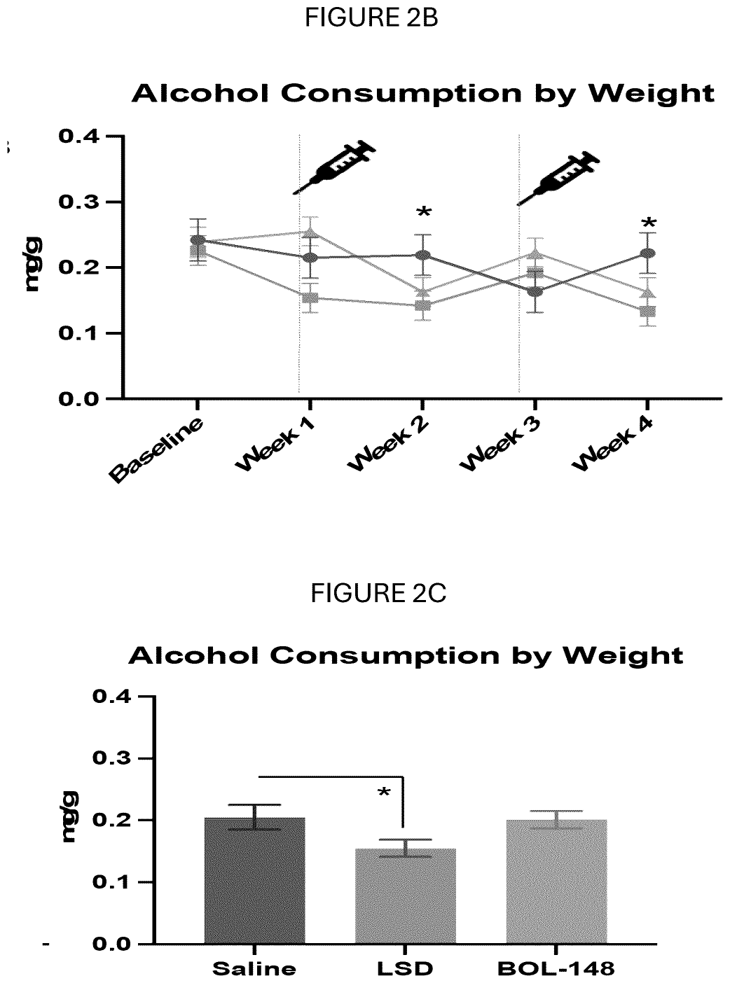

There was a main effect of Treatment for ethanol consumption (FIGS. 2A-2C: mg total ethanol consumed/mg of body weight: F(2,25)=3.50, p=0.046), planned contrasts revealed a reduction in ethanol consumption in LSD-versus Saline-treated groups (Saline vs. LSD: mean difference=0.050 (95% CI: 0.000-0.099); df=25; p=0.049), but not in BOL-versus Saline-treated mice (Saline vs. BOL mean difference=0.004 (95% CI: −0.045-0.053); df=25; p=0.877). A significant interaction between Treatment and Week (F(6,475)=2.50, p=0.021) prompted additional post-hoc comparisons between the LSD- and Saline-treated groups at each week of follow-up, which revealed that the ethanol consumption treatment effect for Saline vs. LSD was significant at weeks 2 and 4 (non-injection weeks), and not weeks 1 and 3 (injection weeks; Week 1: mean difference=0.062 [95% CI: −0.013-0.136]; df=140; p=0.106; Week 2: mean difference=0.077 [95% CI: 0.002-0.152]; df=140; p=0.044; Week 3: mean difference=−0.029 [95% CI: −0.104-0.046]; df=140; p=0.442; Week 4: mean difference=0.089 [95% CI: 0.014-0.164]; df=140; p=0.020). Parallel analyses on ethanol consumption by volume (ml ethanol consumed) and weight (mg ethanol consumed) without correction for body weight yielded identical results.

### Total Fluid Consumption

Results for total fluid consumption (mg total fluid consumed/mg of body weight) indicated a main effect of Treatment for total consumption (body-weight-corrected consumption by weight: F(2,25)=7.40, p=0.003). Planned contrasts revealed an overall reduction in fluid consumption in LSD-versus Saline-treated groups (Saline vs. LSD: mean difference=0.167 (95% CI: 0.076-0.258); df=25; p<0.001), but not in BOL-versus Saline-treated mice (Saline vs. BOL mean difference=0.086 (95% CI: −0.006-0.177); df=25; p=0.064). Parallel analyses on consumption by volume (ml total fluid consumed) without correction for body weight yielded identical results.

### Ethanol Preference

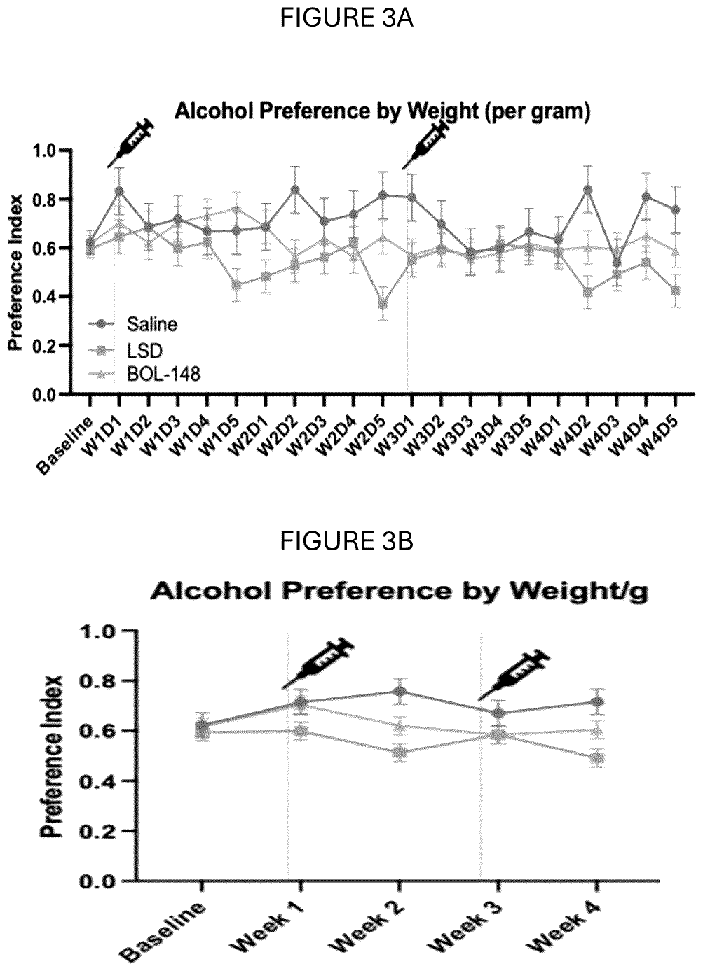

There was a main effect of Treatment for each metric of ethanol preference (FIGS. 3A-3C: volume-based: F(2,25)=7.40, p=0.003; weight-based: F(2,25)=7.41, p=0.003; and body-weight-corrected-based: F(2,25)=7.41, p=0.003).

Planned contrasts to evaluate hypotheses 1a and 2a revealed a reduction in ethanol preference in LSD-versus Saline-treated groups (Saline vs. LSD: mean difference=0.168 (95% CI: 0.076-0.259); df=25; p<0.001), and a non-significant reduction in BOL-versus Saline-treated mice (Saline vs. BOL mean difference-0.086 (95% CI: −0.005-0.178); df=25; p=0.064). Parallel contrasts on ethanol preference by volume (ml ethanol consumed/ml total fluid consumed) and weight (mg ethanol consumed/mg total fluid consumed) without correction for body weight resulted in identical group differences.

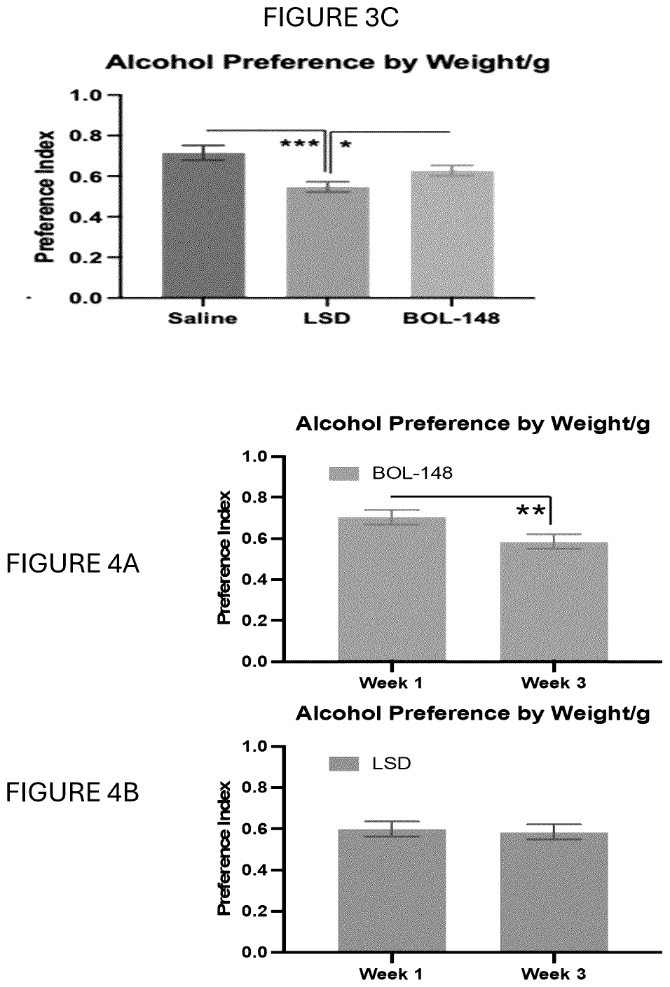

Planned contrasts to evaluate hypothesis 1b did not identify a reduction in ethanol preference in LSD-treated animals from follow-up week 1 to follow-up week 3 (FIG. 4B: week 1 vs. week 3: mean difference=0.014 (95% CI: −0.066-0.094); df=475; p=0.726), but did reveal a reduction in BOL-versus Saline-treated mice (FIG. 4A: week 1 vs. week 3: mean difference=0.119 (95% CI: 0.004-0.039); df=475; p=0.004). Parallel contrasts on ethanol preference by volume (ml ethanol consumed/ml total fluid consumed) and weight (mg ethanol consumed/mg total fluid consumed) without correction for body weight resulted in identical group differences.

Final planned contrasts to evaluate hypothesis 2b identified a reduction in ethanol preference in LSD-versus BOL-148-treated groups (FIG. 1: LSD vs. BOL-148: mean difference=0.081 (95% CI: 0.007-0.156); df=25; p=0.034). Parallel contrasts on ethanol preference by volume (ml ethanol consumed/ml total fluid consumed) and weight (mg ethanol consumed/mg total fluid consumed) without correction for body weight resulted in identical group differences.

### Summary

Results from the study support an effect of LSD on alcohol preference and consumption that becomes apparent immediately, and remains stable for weeks, following a single treatment with LSD (supporting hypothesis 1a). Additional results support the existence of a modest effect of BOL-148 treatment on alcohol preference (partial support for hypothesis 2a). An additive effect was not found for LSD (no support for hypothesis 1b), but an additive effect of BOL-148 was found. Finally, reduced alcohol preference in the LSD-treated was detected, relative to the BOL-148-treated animals (support for hypothesis 2b).

While this study did not find additive effects of two doses of LSD, alcohol preference did significantly decrease from the week after dose 1 to the week after dose 2 in the BOL-148-treated animals. Since BOL-148 is not hallucinogenic/psychedelic in people, weekly/bi-weekly, or more frequent (even daily or multiple time/day) doses of BOL-148 can result in additional reduction in alcohol preference.

Additionally, while there was not a statistically significant reduction in alcohol preference with BOL-148 across follow-up, there was a marginal/near-significant reduction (p=0.064). While traditional results reporting would consider this effect to be in line with the null-hypothesis, extensive literature establishes that: 1) p-values should not be interpreted as measures of effect size (Griffiths and Needleman), 2) discarding effects or classifying them as meaningless based solely on p-values being greater than 0.05 leads to scientific error and apparent irreproducibility (especially in situations where the model is underpowered) [37], and 3) in order to be consistent with use and interpretation recommendations set forth by Fisher, larger p-values should be interpreted as offering some evidence against the null hypothesis (Amrhein, et al., Amrhein, Korner-Nievergelt and Roth]. A post-hoc power analysis revealed that, for each of the proposed hypotheses, 3 groups of 30 animals (total n=90) yields power of 0.9 to detect small effects of approximately d=0.2 at alpha=0.05 with 20 days of repeated measurements (autocorrelation=0.5).

Throughout this application, various publications, including United States patents, are referenced by author and year and patents by number. Full citations for the publications are listed herein. The disclosures of these publications and patents in their entireties are hereby incorporated by reference into this application in order to more fully describe the state of the art to which this invention pertains.

The invention has been described in an illustrative manner and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

1. Bonomo, Y., et al., The Australian drug harms ranking study. J Psychopharmacol, 2019. 33 (7): p. 759-768.
2. Hasin, D. S., et al., Prevalence, correlates, disability, and comorbidity of DSM-IV alcohol abuse and dependence in the United States: results from the National Epidemiologic Survey on Alcohol and Related Conditions. Arch Gen Psychiatry, 2007. 64 (7): p. 830-42.
3. Volkow, N. D. and T. K. Li, Drugs and alcohol: treating and preventing abuse, addiction and their medical consequences. Pharmacol Ther, 2005. 108 (1): p. 3-17.
4. Berglund, M., A better widget? Three lessons for improving addiction treatment from a meta-analytical study. Addiction, 2005. 100 (6): p. 742-50.
5. Anton, R. F. O. M., S. S.; Ciraulo, D. A.; Cisler, R. A.; Couper, D.; Donovan, D. M.; Gastfriend, D. R.; Hosking, J. D.; Johnson, B. A.; LoCastro, J. S.; Longabaugh, R.; Mason, B. J.; Mattson, M. E.; Miller, W. R.; Pettinati, H. M.; Randall, C. L.; Swift, R.; Weiss, R. D.; Williams, L. D.; Zweben, A., Combined pharmacotherapies and behavioral interventions for alcohol dependence: the COMBINE study: a randomized controlled trial. J Stud Alcohol Drugs, 2006. 69: p. 5-13.
6. Rosner, S., et al., Opioid antagonists for alcohol dependence. Cochrane Database Syst Rev, 2010 (12): p. CD001867.
7. Rosner, S., et al., Acamprosate for alcohol dependence. Cochrane Database Syst Rev, 2010 (9): p. CD004332.
8. Abuzzahab, F. S., Sr. and B. J. Anderson, A review of LSD treatment in alcoholism. Int Pharmacopsychiatry, 1971. 6 (4): p. 223-35.
9. Halpern, J. H., The use of hallucinogens in the treatment of addiction. Addiction Research, 1996. 4 (2): p. 177-189.
10. Mangini, M., Treatment of alcoholism using psychedelic drugs: a review of the program of research. J Psychoactive Drugs, 1998. 30 (4): p. 381-418.
11. Dyck, E., ‘Hitting Highs at Rock Bottom’: LSD Treatment for Alcoholism, 1950-1970. Social History of Medicine, 2006. 19 (2): p. 313-329.
12. Grinspoon, L. and J. B. Balakar, Psychedelic drugs reconsidered. 1997, New York: The Lindesmith Center.
13. Krebs, T. S. and P. O. Johansen, Lysergic acid diethylamide (LSD) for alcoholism: meta-analysis of randomized controlled trials. J Psychopharmacol, 2012.
14. Smart, R. G., et al., A controlled study of lysergide in the treatment of alcoholism. 1. The effects on drinking behavior. Q J Stud Alcohol, 1966. 27 (3): p. 469-82.
15. Hollister, L. E., J. Shelton, and G. Krieger, A controlled comparison of lysergic acid diethylamide (LSD) and dextroamphetmine in alcoholics. Am J Psychiatry, 1969. 125 (10): p. 1352-7.
16. Ludwig, A., et al., A clinical study of LSD treatment in alcoholism. Am J Psychiatry, 1969. 126 (1): p. 59-69.
17. Bowen, W. T., R. A. Soskin, and J. W. Chotlos, Lysergic acid diethylamide as a variable in the hospital treatment of alcoholism: a follow-up study. J Nerv Ment Dis, 1970. 150 (2): p. 111-8.
18. Pahnke, W. N., et al., The experimental use of psychedelic (LSD) psychotherapy. JAMA, 1970. 212 (11): p. 1856-63.
19. Tomsovic, M. and R. V. Edwards, Lysergide treatment of schizophrenic and nonschizophrenic alcoholics: a controlled evaluation. Q J Stud Alcohol, 1970. 31 (4): p. 932-49.
20. Moreno, F. A., et al., Safety, tolerability, and efficacy of psilocybin in 9 patients with obsessive-compulsive disorder. J Clin Psychiatry, 2006. 67 (11): p. 1735-40.
21. Carhart-Harris, R. L., et al., Psilocybin with psychological support for treatment-resistant depression: an open-label feasibility study. The Lancet Psychiatry, 2016. 3 (7): p. 619-627.
22. Carhart-Harris, R. L., et al., Psilocybin with psychological support for treatment-resistant depression: six-month follow-up. Psychopharmacology (Berl), 2018. 235 (2): p. 399-408.
23. Grob, C. S., et al., Pilot Study of Psilocybin Treatment for Anxiety in Patients With Advanced-Stage Cancer. Arch Gen Psychiatry, 2010.
24. Ross, S., et al., Rapid and sustained symptom reduction following psilocybin treatment for anxiety and depression in patients with life-threatening cancer: a randomized controlled trial. J Psychopharmacol, 2016. 30 (12): p. 1165-1180.
25. Griffiths, R. R., et al., Psilocybin produces substantial and sustained decreases in depression and anxiety in patients with life-threatening cancer: A randomized double-blind trial. J Psychopharmacol, 2016. 30 (12): p. 1181-1197.
26. 26. Karst, M., et al., The non-hallucinogen 2-bromo-lysergic acid diethylamide as preventative treatment for cluster headache: an open, non-randomized case series. Cephalalgia, 2010. 30 (9): p. 1140-4.
27. Johnson, M. W. and R. R. Griffiths, Psilocybin in smoking cessation: a pilot study, in 118th Annual Meeting of the American Psychological Association. 2010: San Diego, CA.
28. Bogenschutz, M. P., et al., Psilocybin-assisted treatment for alcohol dependence: a proof-of-concept study. J Psychopharmacol, 2015. 29 (3): p. 289-99.
29. Nichols, D. E., Psychedelics. Pharmacol Rev, 2016. 68 (2): p. 264-355.
30. Maurel, S., J. De Vry, and R. Schreiber, 5-HT receptor ligands differentially affect operant oral self-administration of ethanol in the rat. European Journal of Pharmacology, 1999. 370: p. 217-223.
31. Alper, K., et al., LSD Administered as a Single Dose Reduces Alcohol Consumption in C57BL/6J Mice. Front Pharmacol, 2018. 9: p. 994.
32. Halberstadt, A. L. and M. A. Geyer, Characterization of the head-twitch response induced by hallucinogens in mice: detection of the behavior based on the dynamics of head movement. Psychopharmacology (Berl), 2013. 227 (4): p. 727-39.
33. Benneyworth, M. A., et al., Complex discriminative stimulus properties of (+) lysergic acid diethylamide (LSD) in C57Bl/6J mice. Psychopharmacology (Berl), 2005. 179 (4): p. 854-62.
34. Winter, J. C., et al., The Stimulus Properties of LSD in C57BL/6 Mice. Pharmacol Biochem Behav, 2005. 81 (4): p. 830-837.
35. Guenthner, C. J., et al., Permanent genetic access to transiently active neurons via TRAP: targeted recombination in active populations. Neuron, 2013. 78 (5): p. 773-84.
36. Griffiths, P. and J. Needleman, Statistical significance testing and p-values: Defending the indefensible? A discussion paper and position statement. Int J Nurs Stud, 2019. 99: p. 103384.
37. Van Calster, B., et al., Consequences of relying on statistical significance: Some illustrations. Eur J Clin Invest, 2018. 48 (5): p. e12912.
38. Amrhein, V., et al., Retire statistical significance. Nature, 2019. 567.
39. Amrhein, V., F. Korner-Nievergelt, and T. Roth, The earth is flat (p>0.05): significance thresholds and the crisis of unreplicable research. PeerJ, 2017. 5: p. e3544.

What is claimed is:

1. A method of treating substance abuses in an individual, including the steps of:
administering a first dose of 2-bromo-lysergic acid diethylamide (BOL-148) to an individual;
producing an additive effect by administering a second dose of BOL-148 to the individual; and
reducing use of the substance.

2. The method of claim 1, wherein said reducing step includes a step chosen from the group consisting of reducing frequency of use, reducing an amount of substance used, stopping use of the substance, and combinations thereof.

3. The method of claim 1, wherein the substance is chosen from the group consisting of cocaine, nicotine, opiates, alcohol, morphine, and methamphetamine.

4. The method of claim 1, further including the step of treating a behavioral disorder.

5. The method of claim 1, wherein BOL-148 is administered in an amount of 2.1 mg to 210 mg.

6. The method of claim 1, wherein said step of administering a second dose is performed at a time period chosen from the group consisting of an hour, multiple hours, a day, a week, and two weeks after said step of administering a first dose.

7. The method of claim 6, further including the step of administering additional doses of BOL-148 at a time period chosen from the group consisting of hourly, daily, weekly, and bi-weekly.

8. The method of claim 1, wherein said reducing step is caused by non-psychedelic pharmacological properties of BOL-148.
